(19) United States
(12) Patent Application Publication
(10) Pub. No.: US 2023/0290491 A1
(43) Pub. Date: Sep. 14, 2023

(54) SYSTEM AND METHODS FOR DIGITALLY ENHANCING MEDICAL IMAGING CURRENT PROCEDURAL TERMINOLOGY CODES

(71) Applicant: 3DR Labs, LLC, Louisville, KY (US)

(72) Inventors: Judith A. Zakutny, Louisville, KY (US); Stephanie A. Manack, Louisville, KY (US)

(73) Assignee: 3DR Labs, LLC, Louisville, KY (US)

(21) Appl. No.: 18/172,471

(22) Filed: Feb. 22, 2023

Related U.S. Application Data
(60) Provisional application No. 63/318,487, filed on Mar. 10, 2022.

(51) Int. Cl. G16H 40/20; G16H 50/20

(52) U.S. Cl. CPC G16H 40/20; G16H 50/20; G16H 10/60

(57) ABSTRACT

Embodiments disclosed herein may digitally enhance numeric and/or alphanumeric medical imaging codes using one or more enhancement metrics. The enhancement metrics may include, for example, complexity metric, modality metric, patient acuity metric, an add-on score, an additional item score, a technical resource expertise score or equipment capabilities score, one or more of which may be used generate an enhanced resource utilization value resulting in, comprehensive operational and financial performance metrics. The enhanced resource utilization value may more accurately reflect the actual performance medical imaging facilities used as the medical imaging industry standard compared to the conventional systems.

### CROSS-REFERENCE TO RELATED APPLICATION(S)

The present application claims priority to U.S. Provisional Patent Application No. 63/318,487 titled “SYSTEMS AND METHODS FOR DIGITALLY ENHANCING MEDICAL IMAGING CURRENT PROCEDURAL TERMINOLOGY CODES,” and filed Mar. 10, 2022, the entirety of which is incorporated by reference herein.

### BACKGROUND

Medical imaging procedures are generally recorded with one or more pre-determined numeric and/or alphanumeric codes. For example, the Centers for Medicare and Medicaid Services (CMS) yearly publishes Current Procedural Terminology (CPT®) codes as standards for recording different medical imaging exams/procedures. When a patient visits a medical facility for an imaging exam/procedure (e.g., an X-ray), the database record of the exam/procedure may include one or more CPT® codes, identifying the imaging exam/procedure. The database with the CPT® codes may subsequently be used for billing, technical workload analysis, resource utilization, etc. Institutions have also developed other approaches, generally to supplement the CPT® codes for technical workload documentation.

There are, however, multiple technical shortcomings with solely utilizing the conventional standard of the pre-determined alphanumeric codes in medical imaging for technical workload analysis. Medical imaging includes a plethora of exams/procedures ranging from X-rays to CTs to MRI scans, each involving different intricacies, technology, different level of expertise, different levels of personnel involvement, patient acuity, etc.; and the pre-determined numeric and/or alphanumeric codes fail to capture this complexity. Therefore, as detailed in the paragraphs below, downstream analysis (e.g., benchmarking, operational and financial performance) based solely on the count of the pre-determined numeric and/or alphanumeric codes may not accurately reflect the level and complexity of work performed by the technologist in imaging exams/procedures.

The standard numeric and/or alphanumeric code based recording systems generally treat each medical imaging exam/procedure as one unit—without accounting for the variations as described above—and therefore produce inaccurate results. For example, a standard chest x-ray may take just a few minutes, while a spine MM can take several hours. By counting each of these medical imaging exams/procedures equally as one unit, the different operational resources (e.g., time, equipment, personnel) required to complete the medical imaging exam/procedure are incorrectly considered equal.

Another source of inaccuracy using standard numeric and/or alphanumeric codes is the use of multiple codes for a single imaging exam/procedure encounter, while only one code may reflect the actual use of operational resources for the encounter. Conventional systems generally count all the entered codes and the total count generated by these systems therefore may not accurately reflect, in the aggregate, operational resources for an encounter. For example, an encounter for a medical imaging exam/procedure may include a guidance code, a sedation code, an exam/procedure code, supply codes and/or a supervision and interpretation code; which may identify non-patient care functions of the exam/procedure and need not be included as codes for determining the use of technical operational resources. In other words, there is generally no correlation between the amount of operational resources used and the number of the numeric and/or alphanumeric codes. Therefore, the multiple codes may artificially inflate the total calculation.

Furthermore, “add-on” and/or “additional site” exams/procedures that are part of medical imaging exams/procedures are generally assigned their own numeric and/or alphanumeric codes in conventional systems. The add-on or additional-site exams/procedures may include the same procedure but at different areas of the body, e.g., same biopsy procedures for tissues at two different sites. These add-ons or additional sites having their own numeric and/or alphanumeric codes also results in an inaccurate record of the use of operational resources. For example, the operational work to perform a biopsy may include setting up the room, gathering the supplies, positioning and prepping the patient, performing the actual biopsy, post-biopsy care, and the associated administrative steps. Performing a biopsy at two body locations for a single patient may not double the operational work—but the conventional numeric and/or alphanumeric codes will still show a doubling of the operational work.

Conventional systems also fail to account for patient acuity. But the operational resources needed to complete a medical imaging exam/procedure on an ambulatory patient who can dress/undress themselves, get themselves on the scanner or table without assistance, and can respond to instructions/follow commands is vastly different than performing the same exam/procedure on a patient who is unconscious, hooked to various monitors, in pain, non-ambulatory, or in medical distress. Furthermore, performing medical imaging exams/procedures on inpatients or emergency patients may require more operational resources than those performed on outpatients.

Some efforts have been made to mitigate these technical problems with the conventional systems, however, these efforts too remain inadequate. For instance, some systems have used physician relative value units (RVU) to measure the operational resources for medical imaging exams/procedures. But RVU is associated with the physician work of interpreting the medical imaging exam/procedure and generating a report and does not correlate with the operational resources used for generating the medical images. In some instances, the database records with the numeric and/or alphanumeric codes were manually modified to account for the different complexities. But it's an extremely labor-intensive process and is virtually impossible to do in a scalable, repeatable, or credible way.

As such, a significant improvement for recording and analyzing use of operational resources during medical imaging exam/procedure encounters is therefore desired.

### SUMMARY

Embodiments disclosed herein may solve the aforementioned technical problems and may provide other solutions as well. The pre-determined numeric and/or alphanumeric medical imaging codes may be digitally enhanced by using one or more enhancement metrics. The enhancement metrics may include, for example, complexity metric, modality metric, patient acuity metric, an add-in or an additional item score, a technical resource expertise score or equipment capabilities score, one or more of which may be used generate an enhanced resource utilization value for operational and financial performance metrics. The enhanced resource utilization value may more accurately reflect the actual resource utilization and can be used as a consistent measure across the medical imaging industry.

In an embodiment, a processor implemented method of determining resource utilization of multiple medical imaging facilities may be provided. The method may include receiving a plurality of pre-determined numeric and/or alphanumeric medical imaging codes associated with a plurality of medical imaging encounters at a plurality of medical imaging facilities; retrieving one or more enhancement metrics for the received pre-determined numeric and/or alphanumeric medical imaging codes; weighting resource utilization associated with the plurality of pre-determined numeric and/or alphanumeric codes using one or more enhancement metrics to generate enhanced resource utilization values; and causing a display of a dashboard of multiple operational and financial metrics based on the enhanced resource utilization.

In another embodiment, a system may be provided. The system may comprise at least one processor; and a computer-readable non-transitory storage medium storing computer program instructions that when executed by at least one processor causes the one processor to perform operations comprising: receiving a plurality of pre-determined numeric and/or alphanumeric medical imaging codes associated with a plurality of medical imaging encounters at a plurality of medical imaging facilities; retrieving one or more enhancement metrics for the received pre-determined numeric and/or alphanumeric medical imaging codes; weighting resource utilization associated with the plurality of pre-determined numeric and/or alphanumeric codes using one or more enhancement metrics to generate enhanced resource utilization values; and causing a display of a dashboard of multiple operational and financial metrics based on the enhanced resource utilization.

In yet another embodiment, a computer-readable non-transitory storage medium storing computer program instructions may be provided. The computer program instructions that when executed cause operations comprising receiving a plurality of pre-determined numeric and/or alphanumeric medical imaging codes associated with a plurality of medical imaging encounters at a plurality of medical imaging facilities; retrieving one or more enhancement metrics for the received pre-determined numeric and/or alphanumeric medical imaging codes; weighting resource utilization associated with the plurality of pre-determined numeric and/or alphanumeric codes using the one or more enhancement metrics to generate enhanced resource utilization values; and causing a display of a dashboard of multiple operational and financial metrics based on the enhanced resource utilization.

### BRIEF DESCRIPTION OF DRAWINGS

FIG. 1 illustrates an example system for digitally enhancing medical imaging codes, according to some embodiments of this disclosure.

FIG. 2 illustrates a flow diagram of an example method of determining resource utilization of multiple medical imaging facilities, according to some embodiments of the present disclosure.

FIG. 3 illustrates an example dashboard, according to some embodiments of this disclosure.

FIG. 4 illustrates another example dashboard, according to some embodiments of this disclosure

FIG. 5 illustrates another example dashboard, according to some embodiments of this disclosure

### DESCRIPTION

Embodiments disclosed herein may digitally enhance numeric and/or alphanumeric medical imaging codes using one or more enhancement metrics. The enhancement metrics may include, for example, complexity metric, modality metric, patient acuity metric, an add-in score, an additional item score, a technical resource expertise score or equipment capabilities score, one or more of which may be used generate an enhanced resource utilization value. The enhanced resource utilization value may more accurately reflect the actual resource utilization at multiple medical imaging facilities compared to conventional systems.

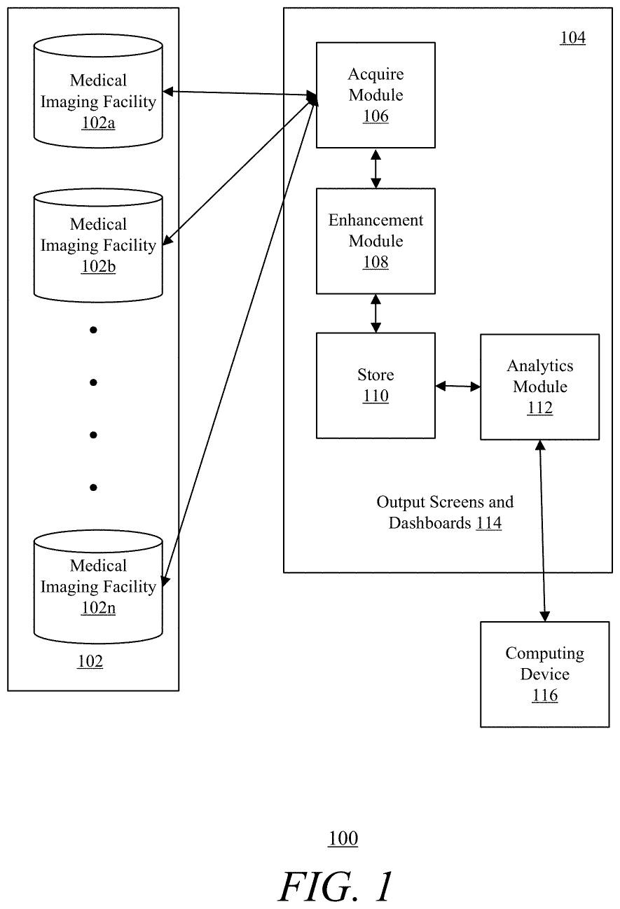

FIG. 1 illustrates an example system 100 for digitally enhancing medical imaging codes, according to some embodiments of this disclosure. It should be understood that the components of the system 100 shown in FIG. 1 and described herein are merely intended as illustrations and systems with additional, alternative, and fewer number of components should also be considered within the scope of this disclosure.

As shown, the system 100 may include one or more healthcare data sources such as medical imaging facilities 102 that may provide data ingested to the system 100. The imaging facilities 102 may be part of the system 100 or may be remote from the system 100 and accessed over a communications path, such as the Internet, Ethernet, a wireless network, a computer network, and the like. As shown, the imaging facilities 102 comprising several different medical imaging facilities: medical imaging facility 102a, medical imaging facility 102b, . . . , medical imaging facility 102n. Each of the medical imaging facilities 102 may perform different types of medical imaging exams/procedures such as X-ray, MRI, CT, biopsy, and the like. Each of the medical imaging facilities 102 may include computing devices and/or database devices that may interact with the medical imaging equipment to receive medical imaging data. The computing devices and/or database may further be used by personnel in the medical imaging facilities 102 to enter numeric and/or alphanumeric medical imaging codes for the corresponding medical imaging exams/procedures. The medical imaging facilities 102 should be understood to include any type of clinical/medical setting, including but not limited to, hospitals, outpatient clinics, mobile clinics, home health visits, health kiosks, trauma centers, first-aid clinics, etc.

In some embodiments, the numeric and/or alphanumeric medical imaging codes entered (e.g., by medical imaging personnel) in the medical imaging facilities 102 include CPT® codes published by CMS. In other embodiments, the numeric and/or alphanumeric medical imaging codes entered by be proprietary codes or facility specific codes. In yet other embodiments, the numeric and/or alphanumeric medical imaging codes may be a combination of the CPT® codes and the proprietary of facility specific codes. Regardless of the types of the codes, the entered codes are standard based on the method used, and therefore may not necessarily reflect the complexity of the medical imaging exams/procedures. Downstream analytics using just these codes are prone to be inaccurate or incorrect. To mitigate these problems, the medical imaging codes are digitally enhanced within the system 100, using the backend component (or platform) 104.

In some examples, data could come directly from the modality equipment such as the actual start and end times of exams. This data would be used for ongoing updates of average exam times.

The backend component 104 may be coupled to the medical imaging facilities 102 over corresponding communication paths. The communication paths may include any kind of wired or wireless communication through any type of packet switching protocols (e.g., the Internet) or circuit switching protocols (e.g., telephony). For example, the backend component 104 may be implemented using one or more computing resources, such as server computers, database server computers, application server computers, blade servers, data center computing resources, cloud-based computing resources that include one or more processors and memory. The modules 106, 108, and 112 may be implemented using a plurality of lines of instructions/computer code that may be stored in the memory and executed by a processor of the backend component 104 to implement the operations and functions of the backend component 104 as described below. Each of the modules 106, 108, and 110 may be alternatively implemented in hardware and each module may be a hardware device that performs the operations and functions of each module as described below. A store 110 of the backend component 104 may be storage for system data, digital logic that is part of the enhancement module 108 that is used to process the incoming medical imaging codes, the logic used by the analytics module 112 that performs the analysis of the healthcare data and generates the operational and financial outputs screens and the dashboards 114, and/or performs any type of storage functionality within the backend component 104. The store 110 may be implemented using hardware (such as a database server, a hardware data storage system, a storage device) or software (such as a software database system or a software based data storage system, or cloud storage services). In some examples, raw data is also stored in 110 in addition to the data after the analysis is performed. This raw data can be used for further artificial intelligence analysis or machine learning for further refinements of the complexity model.

The acquire module 106 may include an interface that may receive/acquire data from each of the medical imaging facilities 102. The acquire module 106 may include, for example, an application programming interface (“API”) that allows the acquire module 106 to gather the data from each of the medical imaging facilities 102. The API may allow for data exchange from a single or multiple imaging facilities. The API may define the standardized format the data must be sent in, allowing for automated seamless communication and accounting for different programming languages that may be used in the disparate imaging systems. The API may also translate the disparate data into a comprehensive bundle of information specific to each patient's exam/procedure, such as procedure CPTs®, modality, patient acuity, technologist and associated skill level, exam/procedure start and end date/times, equipment used, etc.

Alternatively, the acquire module 106 may have other mechanisms (such as to receive batched data or other forms of the data) to gather/poll each medical imaging facility 102 and ingest the data for that particular medical imaging facility (with its possibly unique format) for processing and analysis. The acquire module 106 may be a single acquire module that receives the data from each of the medical imaging facilities 102, or alternatively, the acquire module 106 may include an acquire module 106 for each different medical imaging facility 102, wherein each acquire module 102 may be optimized to receive and process the incoming data from the particular medical imaging facility 102. It should be understood that, to acquire the data, the acquire module 106 may communicate with one or more computing devices within the corresponding medical imaging facility 102.

As described above, the data received by the acquire module 106 from the medical imaging facilities 102 may generally include numeric and/or alphanumeric codes corresponding to the imaging exams/procedures at the medical imaging facilities 102. This is, however, just but an example of the type of received data. The acquire module 106 may receive, and the backend component 104 process, any type of data from the from the medical imaging facilities 102, data such as the patient identification information, and date and time of the medical encounters, etc. In some examples the data may include the actual CPT codes, patient type, number of Modality specific scanners, the number of FTE's per shift, etc., that would be used in the benchmarking module. In addition to performing an analysis of the medical imaging numeric and/or alphanumeric codes, the backend component 104 may perform analyses of the other pieces of data/information as well.

The enhancement module 108 may digitally enhance the received medical imaging numeric and/or alphanumeric codes using one or more embodiments disclosed herein. Particularly, the enhancement module 108 may use enhancement algorithms to add more specific complexity metrics to the received medical imaging numeric and/or alphanumeric codes such that the operational resources required for performing medical imaging exams/procedures at the medical imaging facilities 102 may be more accurately determined. In some examples, Artificial Intelligence (AI) or machine learning may be used as the enhancement algorithms. This more accurate determination may further enhance the downstream analytics such as data aggregation, dashboard generation, predicative analysis, etc. In some examples, the store may not include rules but the enhancement module 108 may apply complexity rules.

In some embodiments, the enhancement module 108 may use metrics such as complexity, modality, patient acuity, add-on, additional-site, technical resource expertise score or equipment capabilities codes to digitally enhance the medical imaging numeric and/or alphanumeric codes received/acquired by the acquire module 106. These metrics are just but some examples, and any type of metric used to enhance the received medical imaging numeric and/or alphanumeric codes should be considered within the scope of this disclosure.

The complexity metric may be used to represent complexity of the medical imaging exam/procedure. In some embodiments, the complexity metric may include, for example, an average time needed to perform the corresponding exam/procedure. For instance, an average time for an arm x-ray may be just a few minutes—the patient's arm just has to be positioned appropriately on the equipment, x-ray images are taken almost instantaneously, and the images are digitally generated shortly thereafter. At the other end, an MRI may take several hours: the patient may have to be injected with contrast material, the patient may have to be sedated, and the patient may have to be in the actual MRI machine itself for several hours. The complexity metric may, therefore, capture that each imaging exam/procedure may be different with different time demand on the medical imaging personnel and equipment. For example, the complexity metric may be smaller for an x-ray exam/procedure compared to an MM exam/procedure.

The complexity metric may generally be pre-calculated and stored at the store 110. The pre-calculation may be based on statistical analysis of different medical imaging exams/procedures. The complexity metric, however, may be continuously improved as the volume of data analyzed grows and new information is learned through the use of AI and/or machine learning. For instance, for the complexity metric based on average time for a medical imaging exam/procedure, the average time may be continuously refined—the average time, for example, may decrease due to technical improvements in the equipment and/or clinical advancements. The average time may further be segmented based on the experience level of the personnel—personnel with a higher experience level may have a lower average time and therefore a lower complexity score than personnel with a lower experience level, such as a student technologist or a medical resident. It should further be understood that the average time and its segmentation thereof based on the experienced level are just but a few examples of the complexity metric and other measures of complexity should also be considered within the scope of this disclosure. For example, a level of expertise required for a medical imaging exam/procedure may also be the basis of the complexity metric, with a higher level of expertise corresponding to lesser complex complexity score and vice-versa.

Factors that go into generating a complexity score may include, in any combination, but are not limited to an average time it takes the technologist to perform all parts of the exam/procedure by modality as the basis for the complexity factor. For example, if the average time it takes to perform a CT is 22 minutes, the base complexity factor is then 2.2 (22/10).

Then, in the example, a differentiator between exams (traditional diagnostic imaging exam such as a CT Brain or X-Ray of the spine) and procedures (more of a therapeutic procedure such as a biopsy under CT guidance, or an Interventional procedures such as an embolism) may also be made and applied.

The modality metric may reflect the type of the medical imaging exam/procedure. For example, the modality in radiology may include different types of exams/procedures such as X-ray (including Bone Densitometry), Ultrasound, CT, MRI, Nuclear Medicine, Positron Emission Testing (PET), Mammography, Interventional Radiology (IR), and the like. The modality metric in conjunction with the type of exam/procedure may reflect the complexity of the exam/procedure by weighting more complex exam/procedures heavier than the less complex exams/procedures. For example, an X-ray may have a lower modality score than an MRI scan.

The modality metric may also generally be pre-calculated and stored at the store 110. The pre-calculation may be based on the known complexity of the exam/procedure—a multi-step process may have a higher modality score compared to a process with lesser number of steps. The modality metric may also be based on the type of equipment. A more sophisticated and more clinically advanced medical equipment may have a higher modality score than a relatively less sophisticated and less advanced medical equipment. As with the complexity metric, the modality metric may not necessarily be static. As the volume of data grows and technological and/or clinical advancements per modality occur, the backend component 104 may continuously refine the modality score based on the newer analysis cycles.

The patient acuity metric may generally be based on the condition of the patient. For example, an ambulatory patient who is able to follow instructions may have a lower patient acuity score compared to a patient who is unconscious. The operational resource utilization therefore may be heavier for patients with the higher acuity scores. In some embodiments, the patient acuity metric may be predetermined and stored on the store 110. For example, the stored patient acuity metric may comprise different scores for different levels of acuity, e.g., fully ambulatory with a low acuity score, partially ambulatory with a medium acuity score, non-ambulatory with a high acuity score. As with the other metrics, the acuity matrix may also be continuously refined as the amount of data (and its analysis) grows. This refinement could consist of further breakdown of levels within the three main patient acuity scores of fully, partially and non-ambulatory such as patients on a ventilator may have a higher level score versus a patient confined to a bed within the non-ambulatory acuity level.

The add-on or additional-site scores may reflect that the add-on and/or additional-site exams/procedures do not necessarily double the operational resources for a single exam/procedure. Based on the complexity of the original exam/procedure, the add-on and/or additional-site scores may weigh the original score with an added fraction to incorporate the corresponding add-on and/or additional-site exam/procedure. For example, the add-on and/or additional-site exam/procedure may be weighted as 50% of the original exam/procedure and therefore the total weight for the entire exam/procedure may be 150% (i.e., the original exam/procedure being weighted by a factor of 1.5). As with the other metrics, the add-on and additional-site scores may be continuously refined as the data and analysis thereof grows.

In some examples, a process of calculating the add-on score or additional-site score may include an assigned 0.5 multiplier; and specimen radiography codes may include an assigned 0.5 multiplier.

A procedure multiplier of 2 may be assigned in all modalities except Interventional (IR) when a procedure is performed. In some examples, IR exams are procedures therefore, no procedure multiplier is assigned. Inpatient and emergency patients may be assigned an 25% complexity of the base complexity value. Bill Only Codes refer to guidance, supervision & interpretation (S&I), and non-patient care functions and therefore, receive a complexity factor of 0.

In some examples, AI, machine learning, or other algorithms may be used in these examples as part of the analysis to produce predicative outcomes.

The analytics module 112 contains the rules used in the generation of each of the output and dashboard screens. The analytics module queries the data from the store module 110. The data which has the complexity metrics applied in the Enhancement module 108 is then mined using algorithms. These algorithms not only analyze the data and associated trends, but also provide predicative analysis for future trending, informed conclusions and decision intelligence. Models are created from these data sets into output screens and dashboards. Algorithms are used to highlight variances or areas needing attention. Predefined rules are used to display the data in each output screen and dashboard. In some examples, the store may store data, and any rules would be applied in the enhancement module 108 and/or the analytics module 112.

The output screens and dashboards 114 generated by the analytics module 112 may be accessed and displayed on one or more computing devices 116 that can access the backend component 104 over a communications path such as a computer network, the Internet, Ethernet, a wireless network and the like. In one implementation, each computing device may execute a browser application and communicate/exchange data with the backend component 104 using a HTML protocol and the backend component 104 may have a web server that generates the HTML code that may be sent to each computing device. However, the system may also be implemented as cloud based services and then the computing device 116 may access the backend component 104 in a different manner that is known in the art. Thus, the system 100 is not limited to any particular way in which the backend component 104 and the computing device 116 interact with each other. Each computing device 116 may include a processor-based device with memory, persistent storage, a display and wireless or wired communications circuits. For example, each computing device 116 may be a smartphone device, such as an Apple iPhone or Android operating system based device, a personal computer, a tablet computer, a laptop computer and the like.

The screens and dashboards 114—generated by the analytics module 112 and viewed on the computing device 116—may include, but are not limited to an optimization analytics dashboard, a benchmarking dashboard, and a scheduling optimizer dashboard. It should also be understood that these are just some example names of the dashboards and dashboards with other names should also be considered within the scope of this disclosure.

The optimization analytics dashboard may comprise a real-time or near real-time dashboard showing the analytics of an imaging facility 102. Based on the numeric and/or alphanumeric medical imaging codes (and/or any other type of information) by the acquire module 106 and then enhanced by the enhancement module 108, the analytics module 112 may—in the optimization analytics dashboard—show productivity, personnel utilization, and equipment utilization. To that end, various analytical outputs may be provided in this dashboard, some of which are detailed below.

A complexity model analytical output may indicate the complexity model (e.g., complexity metric, modality metric, etc.) used to digitally enhance the medical imaging codes. This output may also provide (at least a link to) a document describing the complexity model. The complexity model may utilize a multi-tiered application of values based on the numeric and/or alphanumeric medical imaging codes for an imaging exam/procedure. The first factor may be a complexity metric in other words the base average time it takes to perform an imaging exam/procedure. Additionally, a modality metric (the modality used to perform the exam/procedure), a patient acuity metric (the ambulatory state of a patient), an add-in or an additional item score (additional imaging codes), and/or a resource utilization (the expertise of the involved technologist and the equipment capabilities) values may be applied. The application of all these factors provides an overall complexity score which equates to the overall average time it takes to perform these individual exams/procedures. This model may be used in all algorithms to accurately determine current and predict future resource utilization for operational and financial performance. A multi-source scorecard/report output may display a summary of current and month-over-month and year-over-year billed volume along with additional time frame options, complexity values, personnel, and productivity trends. In this output, the actual performance may be juxtaposed with predicted trends (e.g., budgeted trends). An operational dashboard output may comprise a customizable, permission-based dashboard view of operational data from multiple imaging facilities 102. An hourly volume trend output may show the operational volume trend of different modalities of medical imaging, broken down by different factors (e.g., by site, shift, outpatient, inpatient, emergency). This output may also show year-over-year trending data. A volume variance analysis output may show a comparative analysis of month-over-month, year over year exam/procedure volumes, e.g., by site, modality, percentage change, variance over time, etc. This output may also include a goal and a percentage variation from a goal.

A personnel detail output may show personnel spend of a medical imaging facility 102 broken down by modality and individual personnel against the budget. A productivity detail output may show several metrics related to the efficiencies of performing exams/procedures. For example, this output may compare the billed volume of the exams/procedures against personnel spend for the same exams/procedures. A personnel utilization output may show the worked hours by the personnel. A trending output may show the trend of the personal utilization. This output may further show actual full-time equivalent hours with the recommend full-time equivalent hours. In some examples, the output may include viewing the complexity factor total by modality/hour of day/week/month, year over year trend against the total hours worked.

A turnaround time performance output may show a turnaround time for one or more segments, e.g., for compliance requirements. A turnaround time detail display may show turnaround time compliance for the one or more segments with the medical imaging facility 102 detail and the exam/procedure detail. An additional turnaround time output may display other trends for turnaround times, e.g., inputs, outputs, etc. In some examples, the complexity factor may be included as part of the Turn Around Time performance. A billed volume performance output may show a billed volume and any variances. A billed volume detail output may show a billed volume broken down into modality and individual numeric and/or alphanumeric medical imaging codes including the complexity value trending. A personnel performance output may also display the personnel spend compared to an expected spend (e.g., budget) and any variances.

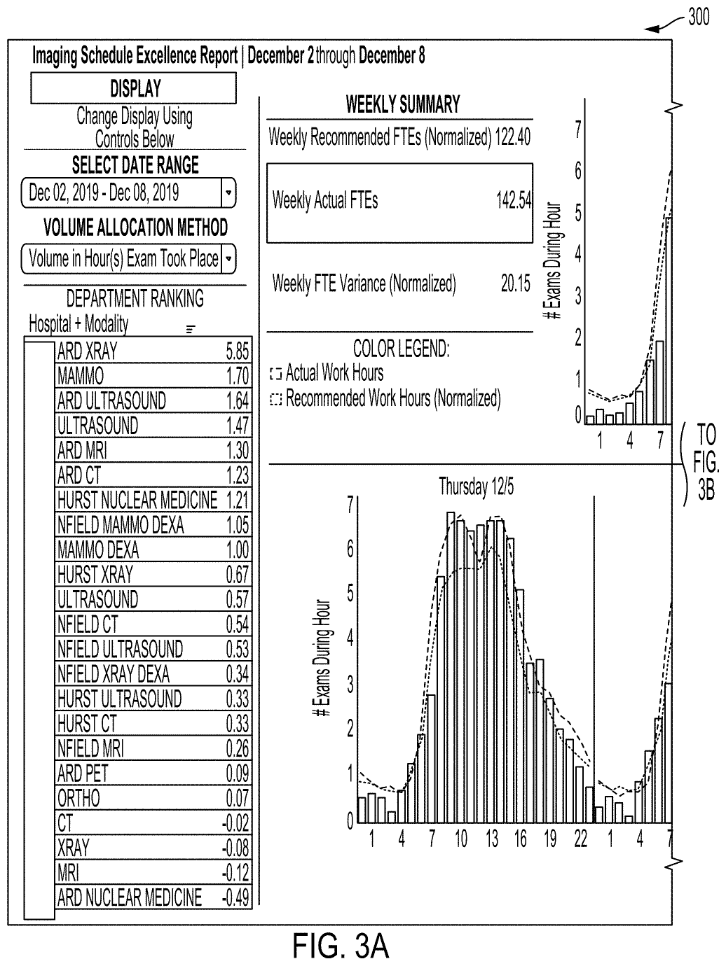

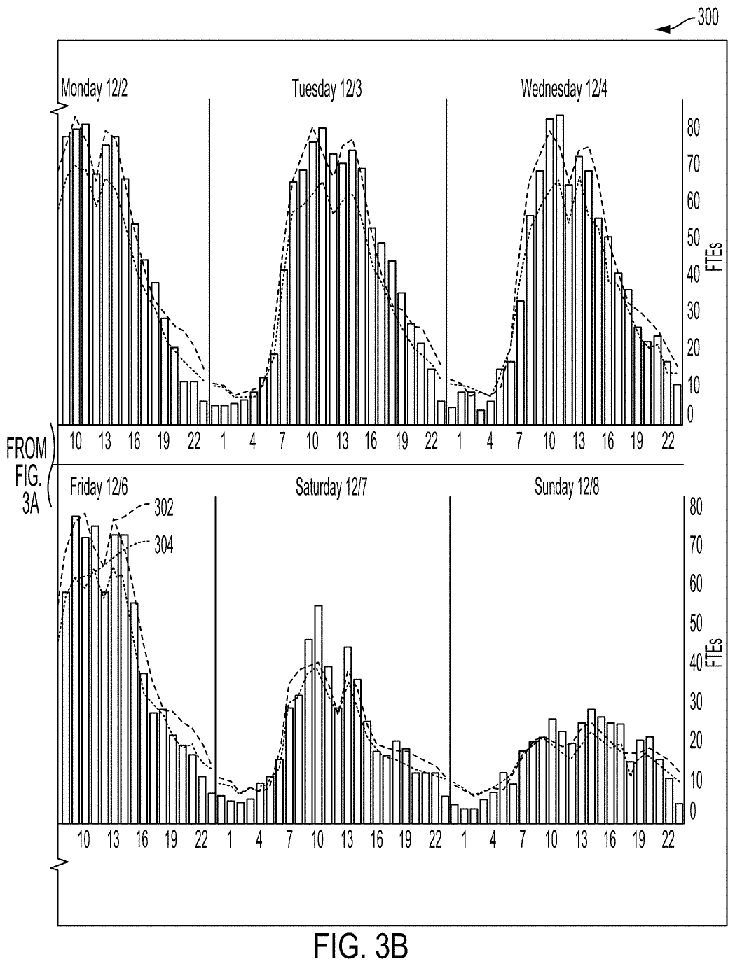

A patient access dashboard output may show patient related factors such as cancellation rate, no show rate, time to third available appointment, call abandonment rate, engagement calls, backlog by modality, etc. This output may be broken down by modality, individual equipment and the site of the medical imaging facility 102. A referrals output may show ordering physician history (e.g., volume) by site, provider, provider group, modality, etc. This output may also show a trend between revenue and modality. An equipment utilization dashboard output may show equipment (or any kind of resource) by room, modality, and location for an adjustable time frame (past, current, and future) and year-over-year and month-over-month comparisons. Again, in some examples, the complexity factor may be included as part of the utilization performance. As another example of a dashboard, the benchmarking dashboard may show a synthesized view of medical imaging specific data. This dashboard may therefore allow for benchmarking the medical imaging practices across different facilities to develop standards to improve operational performance. In some examples, that Imaging specific criteria would be used in order to compare with true like facilities, such as number of CT scanners, number of FTE's per hour, type of facility (rural, academic, etc.), equipment capabilities, etc. As yet another example of a dashboard, the scheduling optimizer dashboard may allow for ideal imaging exam/procedure staffing by modality and hour of the day. The scheduling optimizer dashboard may show a graphical output (an example of which is shown in FIG. 3) of an optimal schedule by hour, modality, and operational resources compared to the actual site and modality schedule. In some examples, AI or machine learning may be used for predicative analysis and modeling of staff schedules.

It should be understood that although the above description describes a rule based system, it should not be considered limiting. Machine learning tools may be used to learn and/or refine the rules. The machine learning tools may also be used for generating the dashboards described herein.

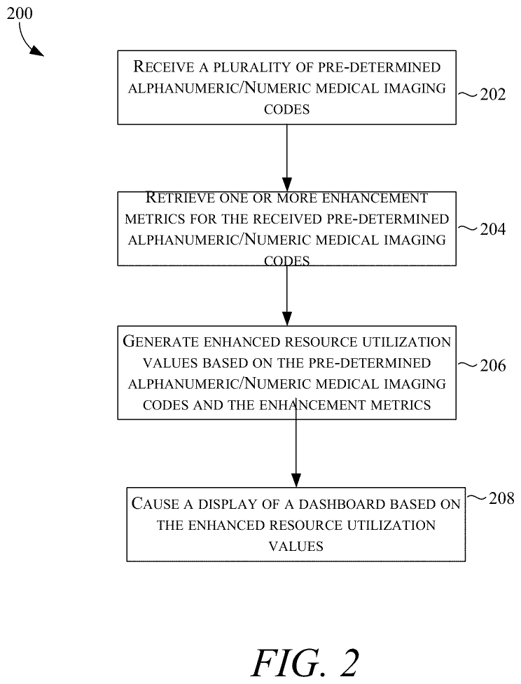

FIG. 2 illustrates a flow diagram of an example method 200 of determining resource utilization of multiple medical imaging facilities, according to some embodiments of the present disclosure. The method 200 is shown has having steps 202, 204, 206, and 208; however, these steps are shown merely as examples and should not be considered limiting. Methods with additional, alternative, or fewer number of steps should be considered within the scope of this disclosure.

At step 202, a plurality of pre-determined medical imaging codes may be received. The pre-determined medical imaging codes may be received by a backend component of a system (e.g., backend system 104 of system 100 shown in FIG. 1) from a plurality of medical imaging facilities (e.g., medical imaging facilities 102 shown in FIG. 1).

At step 204, one or more enhancement metrics for the received pre-determined numeric and/or alphanumerical medical imaging codes may be retrieved. The enhancement metrics may have been stored as rules to digitally enhance the calculations associated with the pre-determined numeric and/or alphanumeric medical imaging codes. Accordingly, at step 206, enhanced resource utilization values may be generated based on the pre-determined numeric and/or alphanumeric medical imaging codes and the retrieved enhancement metrics. In particular, the resource utilization associated with the plurality of pre-determined numeric and/or alphanumeric codes may be weighted using the enhancement metrics to generate an enhanced resource utilization values. At step 208, a dashboard may be displayed based on the enhanced resource utilization values. In some examples, additional exam/procedure related data may be received in conjunction with the resource utilization values to generate the dashboard. The dashboard may allow a more accurate aggregate view of resource utilization at the different medical imaging facilities and further allow an optimization of the resource utilization including equipment and personnel. AI and/or machine learning may take into account personnel and exam/procedure schedules, recommended/required minimum staffing parameters along with scheduled and unscheduled equipment downtime allowing for exam/procedure and personnel reallocation decisions.

FIG. 3 illustrates an example dashboard 300, according to some embodiments of this disclosure. The dashboard 300 may be generated by the analytics module 112 and displayed by the computing device 116. That is, the backend component 104 may cause the display of the dashboard on the computing device 116. As described above, the dashboard 300 may be a scheduling optimizer dashboard. The example optimization that may be realized from the dashboard 300 is related to scheduling personnel for medical imaging exams/procedures. In particular, the dashboard 300 shows actual work hours 302 and the recommended work hours 304. The example optimization based on the dashboard therefore may be used to closely plan to the recommended work hours. In some examples, this may be used in conjunction with AI predicative analysis can auto-generate an actual staff schedule per modality.

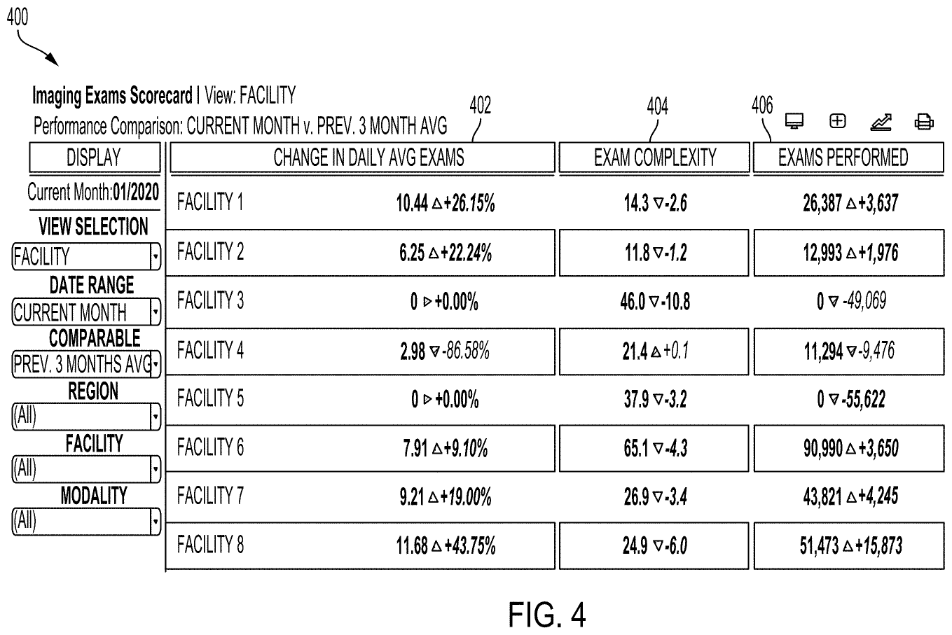

FIG. 4 is an example dashboard 400 showing an imaging scorecard with a plurality of facilities and the change in daily average exams 402, exam complexity 404, and exams performed 406 listed in display for a specified time frame. Additionally, a trending indicator along with the change in value is displayed for each item.

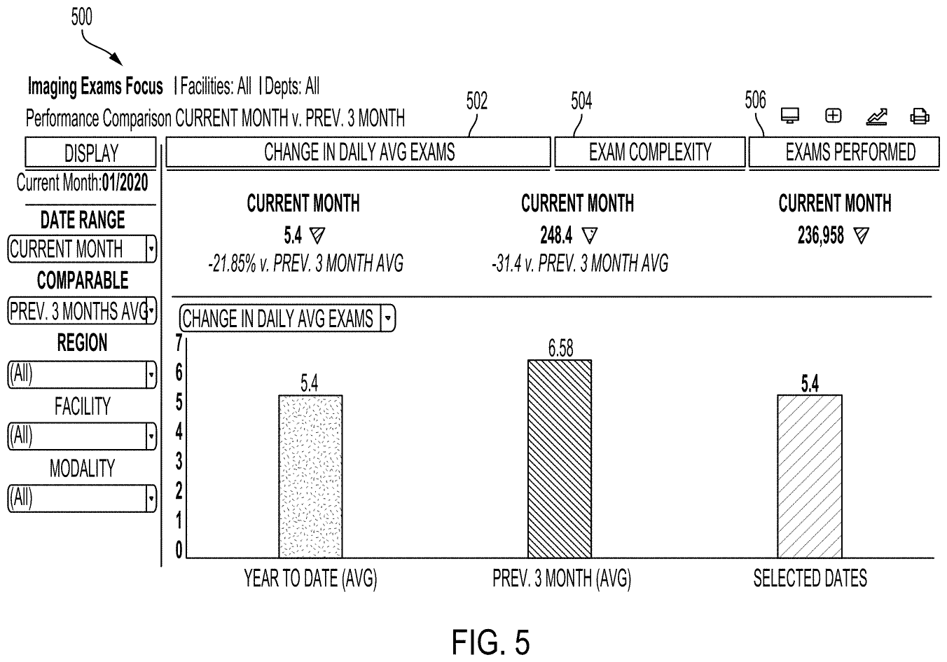

FIG. 5 shows an example dashboard 500 with Imaging exam scores for one set time period and all selected facilities for a change in daily average exams 502, exam complexity 504, and exams performed 506 listed in display. Additionally, a trending indicator along with the change in value is displayed for each item against the previous 3 month average. A visual representation is also displayed for the selected metric in this case the change in daily average exams.

### CONCLUSION

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include an/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers; servers or server computing devices such as routing/connectivity components; hand-held or laptop devices; multiprocessor systems; microprocessor-based systems; set top boxes; consumer electronic devices; network PCs; other existing computer platforms; and distributed computing environments that include one or more of the above systems or devices.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Additionally, the modules can comprise programming instructions transmitted to a general purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise”, “comprising”, and the like are to be construed in an inclusive sense as opposed to an exclusive or exhaustive sense; that is to say, in a sense of “including, but not limited to.” Words using the singular or plural number also include the plural or singular number respectively. Additionally, the words “herein”, “hereunder”, “above”, “below”, and words of similar import refer to this application as a whole and not to any particular portions of this application. When the word “or” is used in reference to a list of two or more items, that word covers all of the following interpretations of the word: any of the items in the list, all of the items in the list and any combination of the items in the list. Furthermore, it is the Applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. A processor implemented method of determining resource utilization using a multi-tiered complexity model applied resulting in automation of performance measures for a single or multiple medical imaging facilities, the method comprising:
receiving a plurality of pre-determined numeric and/or alphanumeric medical imaging codes and other pertinent data associated with an exam/procedure associated with a plurality of encounters at a plurality of medical facilities;
translating the plurality of pre-determined numeric and/or alphanumeric codes using an application program interface (API) that defines a standardized format of data required for an automated seamless communication, accounting for different programming languages used in the multiple medical imaging facilities;
integrating, by the API, the plurality of pre-determined numeric and/or alphanumeric medical imaging codes and the other pertinent data associated with an exam/procedure into a comprehensive bundle of information specific to each patient exam/procedure, from at least one of, exam/procedure current procedure terminologies (CPTs), modality, patient acuity, personnel expertise, exam/procedure start and end date/times;
retrieving one or more enhancement metrics for the received pre-determined numeric and/or alphanumeric medical imaging codes, application of the enhancement metrics is performed on the data;
weighting resource utilization associated with the plurality of pre-determined numeric and/or alphanumeric codes using one or more enhancement metrics to generate enhanced resource utilization values; and
using predefined algorithms and artificial intelligence (AI), data may be inspected, transformed and modeled causing a display of a dynamic, interactive dashboard based on the enhanced resource utilization values.

2. The processor implemented method of claim 1, wherein the one or more enhancement metrics comprise at least one of a complexity metric, modality metric, patient acuity metric, an add-on score, an additional item score, a technical resource expertise score or equipment capabilities score.

3. The processor implemented method of claim 2, wherein the complexity metric comprises a precalculated average time required for a medical imaging encounter corresponding to a numeric medical imaging code.

4. The processor implemented method of claim 2, wherein the modality metric comprises a score corresponding to a type of medical imaging modality utilized to perform the exam/procedure.

5. The processor implemented method of claim 2, wherein the patient acuity metric comprises an ambulatory state of the patient.

6. The processor implemented method of claim 2, wherein each of add-in score or an additional item score comprises a weight for an add-on exam/procedure or an additional item for an original exam/procedure of the medical encounter.

7. The processor implemented method of claim 2, wherein a technical resource expertise or equipment capabilities score compromises a weight for a technical resource expertise or equipment capabilities used to perform the exam/procedure.

8. The processor implemented method of claim 1, wherein a view of the interactive dashboard comprises at least one of an optimization analytics dashboard, a benchmarking dashboard, or a scheduling optimizer dashboard.

9. The processor implemented method of claim 1, wherein the data is stored as raw, without metrics applied and with enhancement metrics applied providing for ongoing use of both aspects of the data in AI or machine learning for constant revisions of predicative outcomes.

10. A system comprising:
at least one processor; and
a computer-readable non-transitory storage medium storing computer program instructions that when executed by the at least one processor causes the at least one processor to perform operations comprising:
receiving a plurality of pre-determined numeric and/or alphanumeric medical imaging codes associated with a plurality of medical imaging encounters at a plurality of medical imaging facilities;
retrieving one or more enhancement metrics for the received pre-determined numeric and/or alphanumeric medical imaging codes;
weighting resource utilization associated with the plurality of pre-determined numeric and/or alphanumeric codes using the one or more enhancement metrics to generate enhanced resource utilization values; and
causing a display of a dashboard based on the enhanced resource utilization values.

11. The system of claim 10, wherein the one or more enhancement metrics comprise at least one of a complexity metric, modality metric, patient acuity metric, an add-on score, an additional item, technical resource expertise score or equipment capabilities score.

12. The system of claim 11, wherein the complexity metric comprises a precalculated average time required for a medical imaging encounter corresponding to a numeric medical imaging code.

13. The system of claim 11, wherein the modality metric comprises a score corresponding to a type of modality utilized to perform an exam/procedure.

14. The system of claim 11, wherein the patient acuity metric comprises an ambulatory state of the patient.

15. The system of claim 11, wherein each add-on score or an additional item score comprises a weight for an add-on exam/procedure or an additional item for an original exam/procedure of the medical encounter.

16. The system of claim 11, wherein each of technical resource expertise score or equipment capabilities score comprises a weight for technical resource expertise score or equipment capabilities score for an original exam/procedure of the medical encounter.

17. The system of claim 10, wherein a view of the dashboard comprises at least one of an optimization analytics dashboard, a benchmarking dashboard, or a scheduling optimizer dashboard.

18. A computer-readable non-transitory storage medium storing computer program instructions that when executed cause operations comprising:
receiving a plurality of pre-determined numeric and/or alphanumeric medical imaging codes associated with a plurality of medical imaging encounters at a plurality of medical imaging facilities;
retrieving one or more enhancement metrics for the received pre-determined numeric and/or alphanumeric medical imaging codes;
weighing resource utilization associated with the plurality of pre-determined numeric and/or alphanumeric codes using the one or more enhancement metrics to generate enhanced resource utilization values; and
causing a display of a dashboard based on the enhanced resource utilization values.

19. The computer-readable non-transitory storage medium of claim 18, wherein the one or more enhancement metrics comprise at least one of a complexity metric, modality metric, patient acuity metric, an add-on score, an additional item score, technical resource expertise score or equipment capabilities score.

20. The computer-readable non-transitory storage medium of claim 19, wherein the complexity metric comprises a precalculated average time required for a medical imaging encounter corresponding to a numeric medical imaging code.

21. The computer-readable non-transitory storage medium of claim 19, wherein the modality metric comprises a score corresponding to a type of modality utilized to perform the exam/procedure.

22. The computer-readable non-transitory storage medium of claim 19, wherein the patient acuity metric comprises an ambulatory state of the patient.

23. The computer-readable non-transitory storage medium of claim 18, wherein the dashboard comprises at least one of an optimization analytics dashboard, a benchmarking dashboard, or a scheduling optimizer dashboard.
